(19) United States
(12) Patent Application Publication
(10) Pub. No.: US 2012/0136388 A1
(43) Pub. Date: May 31, 2012

(54) SURGICAL THREAD WITH SHEATH-CORE CONSTRUCTION

(75) Inventors: Erich Odermatt, Schaffhausen (CH); Ingo Berndt, Tuttlingen (DE); Silke König, Rottweil (DE); Erhard Müller, Stuttgart (DE); Sven Oberhoffner, Weinstadt-Endersbach (DE); Heinrich Planck, Nürtingen (DE)

(73) Assignees: Aesculap AG, Tuttlingen/Donau (DE); ITV Denkendorf Produktservice GmbH, Denkendorf (DE)

(21) Appl. No.: 13/128,164

(22) PCT Filed: Nov. 6, 2009
(86) PCT No.: PCT/EP09/07948

§ 371 Date: Jul. 20, 2011

(30) Foreign Application Priority Data
Nov. 6, 2008 (DE) 10 2008 057 216.0

(51) Int. Cl. A61L 17/04; B28B 11/08; A61L 17/12; A61L 17/10; A61B 17/06

(52) U.S. Cl. 606/222; 606/228; 606/230; 264/145; 264/400

(57) ABSTRACT

A surgical thread includes a polymeric core and a polymeric sheath surrounding the core, wherein the sheath includes barbs for anchoring in human and/or animal, tissues.

### RELATED APPLICATIONS

This is a §371 of International Application No. PCT/EP2009/007948, with an international filing date of Nov. 6, 2009 (WO 2010/052005 A1, published May 14, 2010), which is based on German Patent Application No. 10 2008 057 216.0, filed Nov. 6, 2008, the subject matter of which is incorporated by reference.

### TECHNICAL FIELD

This disclosure relates to a surgical thread having a sheath-core construction and having barbs for anchoring in biological, in particular human and/or animal, tissues, to a surgical kit and also to a method of forming the surgical thread.

### BACKGROUND

Wounds are typically closed in surgery using thread-shaped sutures. These are usually knotted to achieve a secure hold in the tissue. Care must be taken to ensure that the wounds to be closed are always stitched together using an optimal force at the wound edges. If, for example, the wound edges are stitched together too loosely and too non-uniformly, there is a risk in principle of increased scarring or of dehiscence. If, by contrast, the wound edges are stitched together overly tautly, there is a risk that blood flow through the wound edges is restricted, which can give rise to necrotic changes in the surrounding tissue region.

In addition to the risk of possible secondary complications, which may necessitate renewed surgical interventions, there is also always a certain risk that wound closures based on knotted suture will lead to disruptions in the healing process and to unsatisfactory cosmesis for the patients concerned. Another factor is that it is often necessary for several knots, in particular up to 7 knots, to be placed on top of each other to ensure a secure knotted hold. This means that there is a lot of material being introduced into the region of the wound to be cared for, and can more particularly lead to increased foreign-body reactions, particularly in the case of absorbable sutures.

Barbed sutures, which unlike the familiar/conventional threads do not have to be knotted, have also been around for some time. Such knotless or self-retaining sutures usually consist of a monofil thread equipped with barbs along its longitudinal axis. Barbed sutures are described, for example, in U.S. Pat. No. 3,123,077 A, EP 1 559 266 B1, EP 1 560 683 B1 and EP 1 555 946 B1. The barbs are configured on a thread such that the thread can be pulled through the tissue in the direction of the barbs without great resistance and without tissue trauma. When pulled in the opposite direction, however, the barbs deploy and anchor themselves and, hence, also the suture in the surrounding tissue region. This prevents the suture being pulled back through the puncture channel.

The barbs are produced by cutting into a drawn thread material. A problem with this is that, as a result of its having been drawn, the thread material has a narrowed diameter, so that when barbs are cut into such a thread material problems can arise with regard to mechanical strength when the cut into the thread is inaccurate and goes too deep. When the barbs are cut too deeply into the suture, even very small loads can lead to tearing and propagation of the cut sites and hence to a destabilization of the suture. Breakages in the suture can occur in extreme cases.

It could therefore be helpful to provide a surgical thread which is an alternative to the known knotless sutures and which avoids the known disadvantages, more particularly provides adequate security and strength in relation to wound closure. It could also be helpful to provide a method of forming the surgical thread that has distinct processing advantages over the conventional methods of forming knotless sutures.

### SUMMARY

We provide a surgical thread including a polymeric core and a polymeric sheath surrounding the core, wherein the sheath includes barbs for anchoring in human and/or animal tissues.

We also provide a surgical kit including at least one surgical needle and the surgical thread.

We further provide a method of forming the surgical thread, including forming a thread core polymer and a sheathing polymer into a thread having a polymeric sheath-core construction and barbs, and cutting the thread into the sheath.

### BRIEF DESCRIPTION OF THE DRAWINGS

FIG. 1a shows an oblique view of an example of the thread.

FIG. 1b shows a surgical thread cut in the undrawn state.

FIG. 1c shows an example of the thread.

FIG. 1d shows a further examples of the thread.

FIG. 1e shows a further example of the thread.

FIG. 2a shows a further example of the thread.

FIG. 2b shows a view of the cross-sectional surface of an example of the thread.

FIG. 3a shows a further example of the thread.

FIG. 3b shows a view of a cross-sectional area of an example.

FIG. 4a shows a further example of the thread in combination with a surgical needle.

FIGS. 4b,c show an operating technique using the combination, shown in FIG. 4a, of a suture and a surgical needle.

### DETAILED DESCRIPTION

The thread comprises a surgical thread having a polymeric core and a polymeric sheath surrounding the core (sheath-core construction), wherein the sheath includes barbs for anchoring in biological, in particular human and/or animal, tissues.

In other words, we provide a surgical thread, preferably as surgical suture, having a polymeric sheath-core construction, the sheath of which includes barbs. The barbs are generally formed by cuts into the sheath.

The manner in which the polymeric sheath of the thread surrounds its polymeric core may be partial only. Preferably, however, the entire area of the polymeric core is surrounded by the polymeric sheath.

In principle, the polymeric core of the thread and the polymeric sheath may be connected by covalent and/or on-covalent bonds. However, a connection of the polymeric core and the polymeric sheath by non-covalent bonds is preferred.

Furthermore, the polymeric sheath may have a textile or non-textile structure. Preferably, the polymeric sheath has a non-textile structure. In other words, the polymeric sheath is a non-textile sheath in preferred examples.

The polymeric sheath as such may be surrounded by a tubular (hose type) construction, in particular a tubular grid and/or a tubular textile fabric. A tubular knitted fabric is preferred. Thus, there is a beneficial improvement of the linear breaking strength of the thread. The tubular construction is preferably a netting and/or a mesh (tubular meshwork). Preferably, the barbs protrude from meshes of the tubular construction. To that end, the barbs may be cut into the thread prior to drawing, for example. A subsequent drawing of the thread will result in an erection of the barbs to make the barbs stick out from the thread in the vicinity of the meshes of the tubular construction.

The polymeric sheath may have a multilayer, in particular a two-layer, three-layer, four-layer or the like structure. Individual layers of the sheath may be formed from different materials. Thus, different mechanical characteristics of the sheath and thus of the thread as well may be realized. In particular, a tubular construction may be a component of a multilayer structure of the polymeric sheath, as described above for example.

Preferably, the barbs have a barb cut depth (measured perpendicularly from the thread surface) equal to not more than the thickness of the polymeric sheath. More particularly, the barb cut depth may be exactly equal to the thickness of the polymeric sheath. The barbs preferably have a barb cut depth between 10 and 35%, in particular 15 and 30%, of the diameter of the surgical thread.

The polymeric core of the thread is preferably configured to be flexible, more particularly to be flexurally slack. It is particularly preferable when the polymeric core is configured to be more flexible, in particular to be flexurally slacker, than the polymeric sheath. The sheath, by contrast, is preferably configured to be stiff, in particular to be flexurally stiff. Preferably, the polymeric sheath is configured to be stiffer, in particular flexurally stiffer, than the polymeric core. This makes it possible for the thread to have flexible properties on the one hand and stiff barbs on the other. Increased flexibility, in particular flexural slackness, on the part of the thread is particularly advantageous in improving its handling, whereas stiff barbs generally lead to stronger anchoring of the thread in a biological tissue.

The polymeric core may have a lower flexural modulus than the polymeric sheath. Preferably, the polymeric core has a flexural modulus between 200 and 2000 N/mm², preferably 300 and 1200 N/mm². The flexural modulus of the polymeric sheath is preferably between 1000 and 10 000 N/mm², in particular 1500 and 5000 N/mm².

The surgical thread can in principle be formed of any polymeric material suitable for forming sutures. The contemplated polymeric materials may comprise absorbable and/or nonabsorbable polymeric materials. In other words, a partially absorbable thread may be provided. The polymeric core may be formed of a nonabsorbable polymeric material and the polymeric sheath of an absorbable polymeric material, or vice versa, for example. The polymers may in particular be present as homo-, co-, ter- or tetrapolymer or the like. Suitable polymeric materials are, for example, block polymers, in particular block co- or terpolymers. The use of random or alternating co-, ter- or tetrapolymers or the like is similarly possible.

Useful absorbable polymeric materials include, in particular, polymers from the group consisting of polylactide, polyglycolide, polys-caprolactone), poly(para-dioxanone), poly(trimethylene carbonate), poly(hydroxybutyric acid), mixtures thereof, copolymers thereof and terpolymers thereof. Preference is given to co- or terpolymers, preferably block co- or terpolymers, that include at least one monomer from the group consisting of lactide, glycolide, trimethylene carbonate, para-dioxanone, ε-caprolactone and hydroxybutyric acid, in particular γ-hydroxybutyric acid.

Examples of suitable nonabsorbable polymeric materials are polymers from the group consisting polyolefins, polyesters, polyamides, polyurethanes, nylon, silk, cotton, mixtures thereof, copolymers thereof and terpolymers thereof. Polypropylene in particular must be mentioned as suitable polyolefin. Polyethylene terephthalate is an example of a suitable polyester.

The polymeric core of the thread may have a higher melting point, preferably an at least 20° C. higher melting point, than the polymeric sheath. This makes it possible, for example, to cut barbs into the thread under thermal conditions without cutting into the polymeric core of the thread. This preserves properties of the thread core, for example, linear tensile strength, flexibility and/or elongation at break.

The polymeric core of the thread is preferably formed of an absorbable polymeric material, preferably having a glass transition point <30° C. The polymeric core of the thread can be formed in particular of a co- or terpolymer, preferably block co- or terpolymer, comprising at least one monomer from the group consisting of glycolide, lactide, ε-caprolactone, trimethylene carbonate and hydroxybutyric acid. Particular preference is given to Monosyn®, which comprises a triblock terpolymer of glycolide, trimethylene carbonate and ε-caprolactone.

Alternatively, the polymeric core of the thread is formed of a nonabsorbable polymeric material. The nonabsorbable polymeric material may comprise a polymeric material from the group consisting of polyolefins, polyesters, polyamides, mixtures thereof, copolymers thereof and terpolymers thereof. The polymeric material can be selected, for example, from the group consisting polypropylene, polyethylene terephthalate, nylon, silk, cotton, mixtures thereof, copolymers thereof and terpolymers thereof.

The polymeric sheath is preferably formed of an absorbable polymeric material, in particular from the group consisting of poly(para-dioxanone), polys-caprolactone), polyglycolide, polylactide, poly(trimethylene carbonate), poly(hydroxybutyric acid), mixtures thereof, copolymers thereof and terpolymers thereof. Alternatively, the polymeric sheath is formed of a nonabsorbable polymeric material, in particular from the group consisting of polyolefins, polyesters, polyamides, mixtures thereof, copolymers thereof and terpolymers thereof. Examples of nonabsorbable polymeric materials include polypropylene, polyethylene terephthalate, nylon, silk, cotton, mixtures thereof, copolymers thereof and terpolymers thereof.

As mentioned above, the thread may in principle be formed of nonabsorbable polymeric materials only. For example, the polymeric core of the thread may be formed of a polyolefin, in particular polypropylene, and the polymeric sheath of polyethylene terephthalate or polyamide.

The surgical thread, in particular the polymeric core of the thread and/or the polymeric sheath, may be provided with colorings. Preferably, the polymeric core and the polymeric sheath comprise a different color. Thus, a color shade tigering of the thread may be realized, for example. Overall, a different dyeing of the core of the thread and the sheath will primarily improve visibility and in particular handling of the thread for a surgeon. In other words, by this means thread characteristics in relation to practical aspects may be improved.

Preferably, the surgical thread, in particular the polymeric core and/or the polymeric sheath, may have an additivation. Depending on the type of additive, merely the polymeric core of the thread may have additives. This is particularly advantageous, if the barb cut depth at least corresponds to the thickness of the polymeric sheath. In this case, release of additives to the surrounding tissue may be effected at target sites of the thread, where the polymeric core of the thread is exposed due to the cut in barbs. Appropriate additives are in particular biological, medical and/or pharmaceutical agents. Preferred additives are thus selected from the group consisting of antimicrobial, in particular antibiotic, disinfecting, growth-promoting, anti-inflammatory, analgesic and/or odor-controlling active agents. Particularly preferred are growth factors, differentiation factors, recruiting factors and/or adhesion factors. Examples of appropriate growth factors may be selected from the group consisting of fibroblast growth factor (FGF), transforming growth factor (TGF), platelet derived growth factor (PDGF), epidermal growth factor (EGF), granulocyte-macrophage colony stimulation factor (GMCSF), vascular endothelial growth factor (VEGF), insuline-like growth factors (IGF), hepatocyte growth factor (HGF), interleucin-1 B (IL-1 B), interleucin-8 (IL-8), nerve growth factor (NGF), and combinations thereof.

The additives may be cells, like fibroblas ts and/or chondrocytes and/or precursor cells, in particular stem cells, for example. In other words, the thread, in particular the polymeric core and/or the polymeric sheath may be inoculated with cells, particularly body cells, preferably autologous body cells. A thread inoculated with chondrocytes may be applied as meniscus, tendon and/or ligament suture, for example. In other words, the thread is utile in the treatment of meniscus, tendon and/or ligament disorders. Generally, an addition of cells to the thread is beneficial in that substances produced and secreted by the cells will be an active aid in wound healing. Cellular formed collagen may contribute to smoothing of wrinkles, in particular with surgical threads used in plastic surgery, for example. A cellular colonization of the thread will be enhanced in particular as a result of the barbs enlarging and preferably roughening the surface of the thread.

The thread preferably has an elongation at break between 15 and 100%, in particular 30 and 90%. It is particularly preferable when the thread has a linear tensile strength between 100 and 700 N/mm², preferably 150 and 700 N/mm², in particular 200 and 600 N/mm², based on a thread diameter without barbs.

The proportion of the overall volume of the thread which is accounted for by the polymeric sheath may be between 34% and 90% by volume, in particular 55% and 84% by volume. Correspondingly, the proportion of the overall volume of the thread which is accounted for by the polymeric core may be between 66% and 10% by volume, in particular 45% and 16% by volume. The surgical thread may also have a thickness between 0.2 and 1.2 mm, in particular 0.25 and 0.9 mm.

The barbs themselves may in principle be configured in different shapes and geometries. For example, the barbs may have an escutcheon, shield, scale, wedge, spike, dart, V and/or W shape. Preferably, the barbs are pointed/sharp at their distal end.

The barbs may further be configured in principle in different arrangements on the surgical thread. For example, the barbs may have a row-shaped arrangement, an offset arrangement, a zigzag-shaped arrangement, an overlapping arrangement, an offset and partially overlapping arrangement, a spiral or helical arrangement, a random arrangement, or combinations thereof, in the longitudinal and/or transverse direction, preferably in the longitudinal direction, of the thread. Particular preference is given to an arrangement in which the barbs are distributed over the entire surface of the thread, since the thread is in this case particularly strongly anchorable in a biological tissue. Particularly preferred is a spiral or helical arrangement of the barbs on the surgical thread. Furthermore; particular emphasis is given to an offset arrangement of the barbs, wherein the barbs are partially overlapping one another. Such an arrangement may be realized, for example, by forming barbs with a small angular offset and in small intervals between each other on the thread, preferably by shallow cuts into the thread. In such an arrangement, two adjacent barbs form a barb having a twin-tip configuration (“double-acting” barb). Such a twin-tip configuration is primarily advantageous in relation to solid and secure anchoring of the thread in biological tissue.

The surgical thread may include at least one set, in particular two, three or more sets, of barbs. A set of barbs is herein to be understood as referring to an arrangement of barbs on the surgical thread that is congruent in respect of the configuration of the barbs, in particular in respect of barb height, barb length, barb cut depth, tip angle, deploy angle, orientation of the barbs and/or form or shape of the barbs.

It is particularly preferable for the surgical thread to have a so-called “bidirectional” arrangement of barbs. A bidirectional arrangement of barbs is herein to be understood as referring to an arrangement in which the barbs are orientated in two different directions. Preferably, when viewed in the longitudinal direction of the surgical thread, the barbs are formed in a first portion of the thread to face in the direction of a remaining second portion of the thread and in the remaining second portion of the thread to face in the direction of the first portion of the thread. Particularly preferably, when viewed in the longitudinal direction of the surgical thread, the barbs are formed in a first portion of the thread to face the thread midpoint direction and in a remaining second portion of the thread similarly to face in the direction of the thread midpoint. The length of the portions of thread is preferably approximately equal to half the thread length, so that the thread midpoint forms a kind of center of symmetry. In this way, the surgical thread can be pulled at one end through a biological tissue without major resistance up to about the length midpoint of the thread, the barbs deploying when the thread is pulled in the opposite direction and thereby anchor/retain the thread in the tissue without any need for knotting.

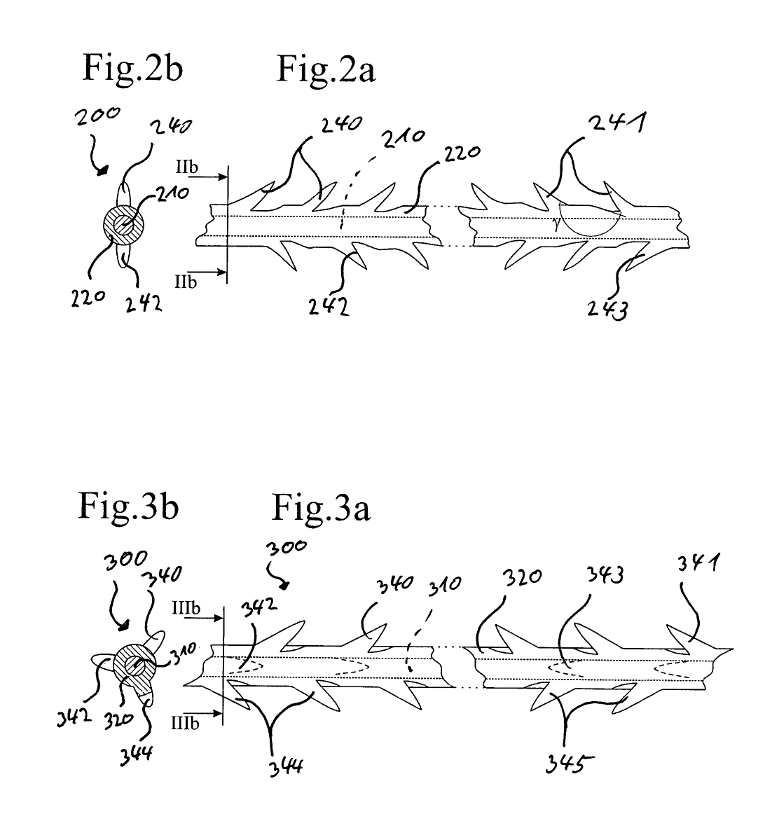

Particularly advantageously, the surgical thread has at least two bidirectional arrangements of barbs on its surface. It is particularly preferable when a first bidirectional arrangement of barbs is formed on the thread surface about 180 degrees, in the circumferential direction of the thread, from and preferably offset with respect to a second bidirectional arrangement of barbs on the thread surface (cf. FIGS. 2a,b). It can further be provided that the surgical thread includes altogether three bidirectional arrangements of barbs. In this case, it is preferable when a first bidirectional arrangement of barbs is formed about 120 degrees, in the circumferential direction of the thread, from and preferably offset with respect to a second bidirectional arrangement of barbs which in turn is formed about 120 degrees, in the circumferential direction of the thread, from and preferably offset with respect to a third bidirectional arrangement of barbs, so that the third bidirectional arrangement of barbs is likewise disposed about 120 degrees, in the circumferential direction of the thread, from and preferably offset with respect to the first bidirectional arrangement of barbs (cf. FIGS. 3a,b).

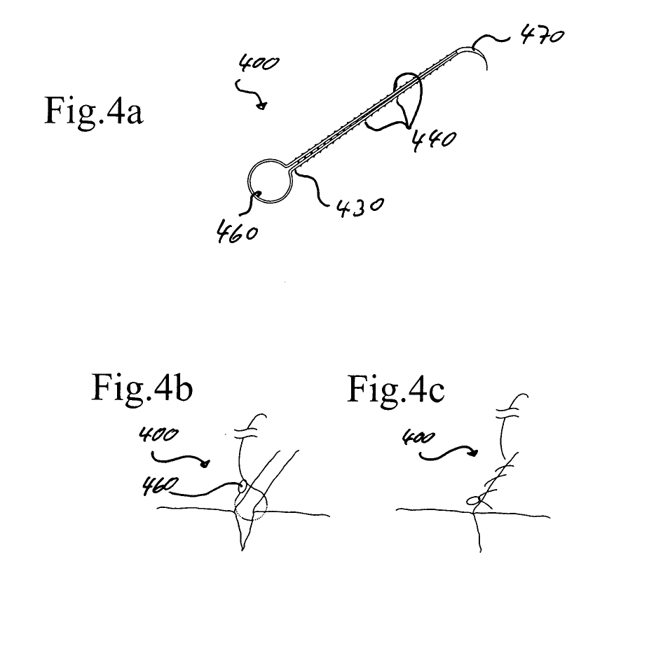

It can further be provided that the surgical thread includes areal regions or portions without barbs. Preferably, the surgical thread has approximately in the region of the thread midpoint an areal portion without barbs. This areal portion may, when considered in the longitudinal direction of the suture, have a length between 0.5 and 5 cm, in particular 1.5 and 3 cm, preferably a length of about 2 cm. As a result, the thread ends can form a loop to lie side by side and preferably be attached to a surgical needle (cf. FIGS. 4a-c). Preferably, the remaining areal portions of the thread have a bidirectional arrangement of barbs, so that, after forming the loop, the barbs point unidirectionally in the direction of the loop. With regard to possible arrangements for the barbs, reference is made to the preceding description.

The barbs may have a so-called “apex angle α” between 13 and 60 degrees, in particular 15 and 40 degrees. It is particularly preferable when the barbs project from the surface of the surgical thread. Preferably, the barbs have a deploy angle β, measured from the cut surface of the barb underside to the cut surface of the thread body or strand, between 12 and 50 degrees, in particular 17 and 45 degrees. The surgical thread can be characterized by a so-called “cut area angle γ ” which is measured from the cut area of the thread strand to the uncut surface of the thread strand. This cut area angle γ can be between 112 and 167 degrees, in particular 120 and 165 degrees.

The barbs, particularly barbs projecting in the longitudinal direction of the thread, can have a mutual spacing between 0.5 and 5 mm, preferably 1.0 and 2.0 mm, measured from barb tip to barb tip.

In general, the surgical thread is in a drawn state. However, it is preferable when the barbs themselves are formed by cuts into the surgical thread in its undrawn state. Because the thread generally decreases in diameter in any subsequent drawing operation, whereas the barbs essentially retain their original shape and size, completely novel, variable barb geometries can be realized. More particularly, the barbs can have mechanical properties, particularly in relation to hardness, flexibility, bendability and elasticity, which differ from the rest of the thread, the so-called thread main body. This makes it possible for the properties of the barbs to be adapted to the properties of the thread, particularly the polymeric core of the thread, in a controlled manner. It is further of advantage when, as a consequence of a drawing operation being carried out on the thread which has been subjected to a cutting action in the undrawn state, portions of the thread which have been subject to cutting and portions of the thread which have not been subject to cutting become more alike with regard to their diameter. As a result, the barbs can no longer completely disappear into the undercuts originally formed by cutting into the thread, which results in improved anchoring in biological tissues. A further advantage concerns the manufacturing operation. In general, an undrawn thread is softer and its machining is therefore simpler and creates less wear on the cutting device. An additional advantage is that the barbs usually deploy automatically, i.e., without auxiliary means, as a result of a subsequent drawing operation. In general, the barbs can be made to deploy synchronously with a subsequent drawing operation.

Preferably, the surgical thread has a circular cross section. The thread preferably has a circular cross section, wherein the proportion of the radius which is accounted for by the polymeric core is preferably between 30 and 90%, in particular 40 and 70%. However, other cross-sectional shapes are also conceivable in principle. For example, the thread may have an oval, triangular or trilobal, square, trapezoidal, rhomboidal, pentagonal, hexagonal, star-shaped or cruciform cross section. Such cross-sectional shapes can readily be realized with the aid of corresponding extrusion dies which can be custom made with any desired cross-sectional shape.

The thread may have a profiled or molded surface, in particular a sinuous or wavelike surface. This may be beneficial regarding the formation of barbs with different structures.

The surgical thread preferably comprises a monofil thread, a monofilament. However, it is also possible in principle for the surgical thread to be a multifilament, in particular a multifilament yarn. It can further also be contemplated that the surgical thread is present as a so-called pseudomonofilament. A pseudomonofilament is a surgical thread having a sheath-core construction wherein the core is formed of a multifilament.

The surgical thread may be pointed/sharpened at one end at least, preferably at both ends such that its penetration through a biological tissue may be facilitated.

At least one end of the thread may be attached to a surgical needle. In the case of the loop-shaped thread already described, generally both ends of the thread are attached to one surgical needle. If, by contrast, the thread has a bidirectional arrangement, it is preferable when the two ends of the thread are each attached to a surgical needle. To attach the thread to the surgical needle, the thread is very generally threaded into a hole in the needle and the needle is subsequently crimped together in the region of the hole.

To avoid puncture channel haemorrhages, it can be provided that the thread has a smaller diameter in the region of its ends than in its remaining regions. In other words, the ends of the thread can have a tapered diameter. It is a particular advantage that such a thread can be combined with a surgical needle which is actually designed for smaller thread diameters. In this way it is possible to achieve an alignment of the thread diameter with the needle diameter. We provide a diameter ratio of needle to thread <2:1, preferably 1:1. As a result, the puncture channel formed by the needle is more fully occupied by the thread regions having the original (untapered) diameter. Preferably, the tapered diameter in the region of the thread ends is equal to the diameter of the polymeric core of the thread. The other regions of the thread preferably have the original diameter (including the thickness of the polymeric sheath). Particular preference is given to a thread whose polymeric core has a diameter which is equal to the diameter of a needle hole and the overall diameter of which (including the thickness of the polymeric sheath) is equal to the needle diameter. To taper the diameter, the thread can be peeled off in the region of its ends. Preferably, it is only the polymeric sheath which is peeled off in the region of the thread ends. To peel the thread, thermal methods can be used, particularly laser techniques. The transition from the original diameter of the thread to the tapered diameter in the region of the thread ends can be formed to be abrupt or continuous, particularly in the form of a gradient. To form a gradual transition, extrusion technology is suitable in particular. The withdrawal speed in extruding a thread can be varied, periodically in particular. This can be accomplished, for example, by modulating the rotary speed of the thread-withdrawing godet. Alternatively, additional godets can be disposed between the extrusion die and the withdrawal godet.

It is particularly preferable when the surgical thread is a surgical suture.

We further provide a surgical kit/set comprising at least a surgical needle and the thread. With regard to further features and details relating to the kit/set, reference is made to the preceding description.

We still further provide a method of forming the thread, wherein a thread core polymer and a sheathing polymer are formed into a thread having a polymeric sheath-core construction and barbs are then cut into the sheath of the thread.

Preferably, the thread having the polymeric sheath-core construction is formed by coextrusion, in particular bicomponent extrusion, of the thread core polymer and of the sheathing polymer. Alternatively, the thread having the polymeric sheath-core construction is formed by a sheathing extrusion, wherein the thread core polymer is sheathed by the sheathing polymer. In this instance, the thread core polymer can be used, for example, as monofilament or multifilament, in particular multifilament yarn, so that sheathing coextrusion can also be used to produce pseudomonofilaments, into the sheath of which barbs are subsequently cut.

The barbs are preferably cut into the sheath to a depth (measured perpendicularly from the thread surface) equal to not more than the thickness, in particular exactly the thickness, of the sheath. The barbs are more preferably cut into the sheath to a depth between 10 and 35%, in particular 15 and 30%, of the diameter of the thread.

The barbs may in principle be cut in the drawn or undrawn state of the thread. It may be contemplated in particular that the barbs are cut into the thread in the undrawn state of the thread and the thread is drawn thereafter. When the barbs are cut in the undrawn state, the barbs may be cut into the undrawn thread in a cut angle θ, relative to the outside surface of the undrawn thread, between 15 and 50 degrees, in particular 20 and 40 degrees. It has emerged that, surprisingly, barbs produced by small cut angles θ can be made to deploy more uprightly by a subsequent drawing of the thread than barbs produced by large cut angles θ. More particularly, the difference between the deploy angle β and the cut angle θ (β-θ) is greater for small cut angles θ than for large cut angles θ. It has further emerged that barbs produced by comparatively large cut angles θ, in particular by cut angles θ≧30 degrees, have more built-up backs. The barb backs in this case generally display a build-up of material, in particular in the form of an accumulation or thickening. This enhances the stiffness, particularly the flexural stiffness, of the hooks, which enhances the anchoring of the thread in biological tissues and hence generally the security of wound closure. In other words, choice of the cut angle θ makes it possible to influence barb geometry and create whichever is the best barb geometry for a particular application.

The barbs may be cut into the thread while the thread is rotated. In accordance with the above explanations, the thread to be treated may be in a drawn or in an undrawn condition. The barbs may also be cut into an undrawn thread, and in a subsequent drawing operation the thread may be twisted simultaneously. Alternatively, the thread may be twisted prior to barb cutting, and again detwisted when the cutting in of barbs is completed. The examples specified in this paragraph are particularly advantageous to produce a radial, in particular spiral or helical, arrangement of barbs on the thread surface.

We can also produce the thread by utilizing a thread core polymer whose melting point is higher, preferably at least 20° C. higher, than the melting point of the sheathing polymer.

Preferably, the barbs are cut into the sheath thermally, in particular in a temperature range below the melting point of the thread core polymer. This contributes to enhanced cut consistency when producing the barbs. Thermal cutting of barbs further has the advantage over purely mechanical cutting, which is similarly possible, that the cut ends in the thread strand which are produced by thermal cutting are less tapered, more particularly less acute, than result from purely mechanical cutting. This can be used to minimize the risk of the thread developing under load a tear starting from the respective cut ends. Thermal cutting of barbs can be performed, for example, with the aid of a cutting wire, more particularly a metal wire, suitable for this purpose. Preference is given to using a heated, particularly an electrically heated, cutting wire. The cutting wire may comprise a fine wire. Preference is given to using a cutting wire having a diameter between 20 and 50 μm. As an alternative to a single cutting wire, it is also possible to use a sheet of cutting wires. It is similarly possible to use a metal grid.

The barbs may be cut into the sheath mechanically, preferably by at least one cutting blade. Customary cutting devices can be utilized. These customarily include a cutting bed, at least one cutting blade and also holding or retaining elements, for example, a vice, chucks, holding or clamping jaws, and the like. The mechanical cutting of barbs particularly preferably utilizes a cutting bed with a groove, the groove being intended to receive the thread—relaxed or twisted—to be cut into. Depending on the depth of the groove, the use of at least one cutting blade allows specific control of the cut depth to which the barbs are cut into the sheath of the thread. This is because the at least one cutting blade is generally configured such that with it only at most the regions of the thread which protrude from the groove can be cut into. This permits improved cutting consistency when producing barbs.

Laser cutting processes are another way of cutting barbs into the sheath. In other words, the barbs can also be cut into the sheath by a laser. Useful lasers include in principle not only gas lasers, for example, CO₂ lasers, but also solid state lasers, for example, Nd:YAG lasers. In general, a suitable laser cutting machine consists of a laser beam source, a beam guide and a usually mobile system of focusing optics (concave mirror or position lens). The beam leaving the beam source is guided either through an optical fiber in the case of an Nd:YAG laser for example, or via a deflecting mirror, in the case of the CO₂ laser for example, to the machining optics which focus the laser beam and thereby produce the power densities required for cutting, which generally range from 10⁶ to 10⁹ W/cm². Appropriate laser cutting processes are well known, so that more far-reaching observations can be dispensed with here.

When the barbs are produced by cutting into the sheath of an undrawn thread, the thread is very generally drawn subsequently. Drawing is preferably effected by heating, in particular in a temperature range between 20 and 80° C. above the glass transition point of the thread. Infrared rays, for example, can be used to produce heat. Drawing can further be carried out in an oven or with the aid of heatable rolls, rollers or godets. Drawing can be carried out continuously or discontinuously. In continuous drawing, the thread is generally guided over a roller or godet system comprising a set of rollers or godets which can have different speeds of rotation. Usually, each subsequent roller has a higher speed of rotation than the preceding roller of the drawing system. In the case of discontinuous drawing, by contrast, the thread is generally clamped between suitable holding or retaining elements, for example, clamping jaws, of a tensioning device and subsequently drawn. To draw the thread it is possible to use a draw ratio between 2.5 and 8, in particular 3 and 5.

After drawing, the surgical thread can be subjected to various post-treatment steps. In general, the thread is for this heat-conditioned in vacuo. This can be used to increase the crystallinity of the thread and reduce its residual monomer content. A further advantage which can be achieved through a post-treatment of the thread relates to the reduced susceptibility to shrinking.

We further provide for the use of the surgical thread as surgical suture, particularly as knotless or self-retaining suture. As already mentioned, the barbs serve to anchor the thread in biological, particularly human and/or animal, tissues. The tissues may comprise, for example, skin, fat, fascia, bone, muscle, organs, nerves, blood vessels, connective tissue, sinews, tendons or ligaments. The surgical thread is preferably employed in plastic surgery, preferably for tightening the skin. For example, the thread is suitable for eyebrow lifts. In addition, however, the thread is also suitable for other surgical indications, particularly for indications in which the use of conventional sutures is made difficult by steric hindrance. For example, the surgical thread can be used in laparoscopic interventions, particularly for retaining meshes, for example, hernia meshes, prolapse meshes or urinary incontinence meshes. A further possible area of use relates to the performance of anastomoses, in particular vascular or intestinal anastomoses.

Altogether, the thread makes it possible to achieve the following advantages in particular:
The sheath-core construction provides for the surgical thread to choose the polymeric materials for the thread core and the sheath independently of each other. This makes it possible, for example, to use a polymeric material having a low flexural modulus for the thread core and a polymeric material having a comparatively high flexural modulus for the sheath. A thread resulting therefrom has comparatively stiff barbs, relative to its core, without the thread becoming altogether too stiff. This is particularly effectively implementable with threads whose polymeric sheath is of low thickness relative to the polymeric core. Improved stiffness for the barbs can additionally or alternatively be achieved by cutting the barbs in the undrawn state of the thread, particularly at cut angles θ≧30 degrees. High stiffness, in particular flexural stiffness, on the part of the barbs provides a general improvement in the anchoring in biological tissues of the thread (“Rawlplug effect”) and hence also increases wound closure security. While conventional barbed sutures are in principle prone to the risk of tearing from the respective cut ends, the thread generally does not give rise to any tearing beyond the boundary between the core and the sheath. When, more particularly, the polymer chosen for the sheath has a lower melting point than the melting point of the polymer for the polymeric core of the thread, barbs can be cut thermally into the thread without injuring or damaging the core of the thread in the process. This, as already mentioned, contributes to enhanced cutting consistency when producing the barbs on the thread.

Further features will become apparent from the following description with reference to examples and figure descriptions in conjunction with features of the drawings. Individual features can be actualized either singly or pluraly in combination with each other.

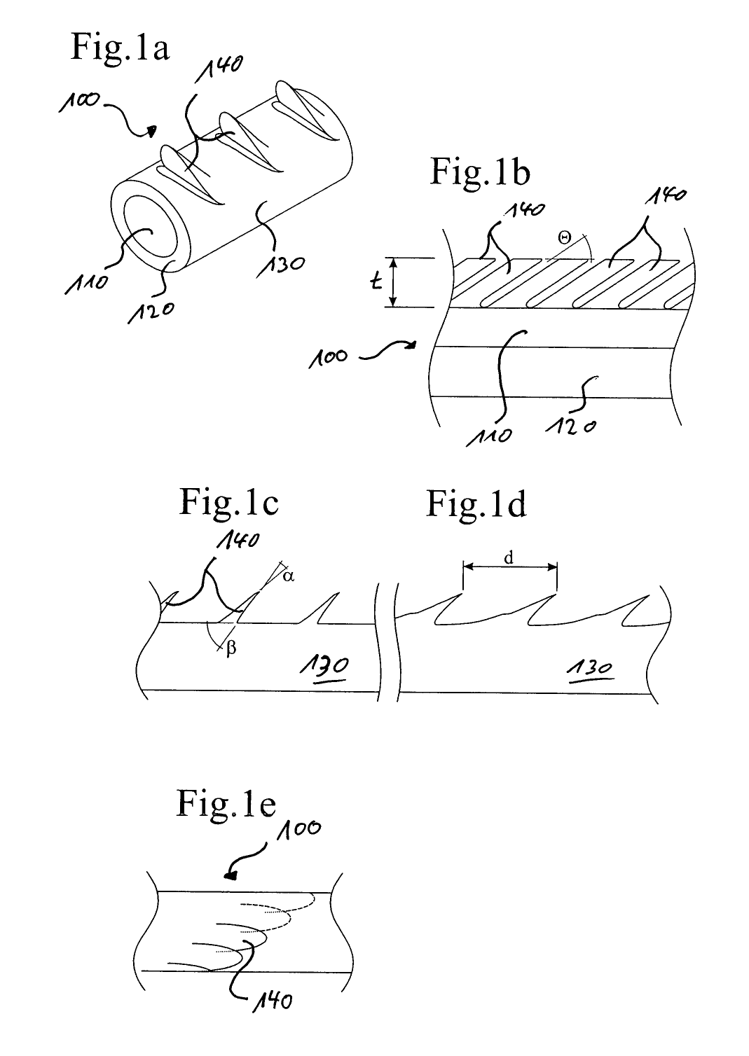

FIG. 1a is a schematic view of thread 100. The thread 100 has a polymeric core 110 and a polymeric sheath 120 surrounding the core 110 (sheath-core construction). The thread 100 further has an elongate main body 130. Barbs 140 have been cut into the polymeric sheath 120 in the longitudinal direction of the thread 100. The polymeric core 110, by contrast, has no cuts.

FIG. 1b is a schematic view of the thread 100 of FIG. 1a in the undrawn state. The barbs 140 have been produced by cuts into the polymeric sheath 120 of the thread 100. The cuts are made at a cut angle θ to a cut depth t (measured as perpendicular from the thread surface).

FIG. 1c is a schematic side view of the thread 100 of FIG. 1a. The barbs 140 are spaced apart by a distance d in the longitudinal direction of the thread 100. The barbs 140 are arranged such that they all face in one direction (unidirectional arrangement). The barbs 140 can be characterized by an apex angle α and a deploy angle β. The apex angle α is to be understood as the angle that results on intersecting an imaginary continuation of the cut surface of the underside of the barb with an imaginary continuation of the back of the barb. The deploy angle β represents the angle that is formed by the cut surfaces of the undersides of the barbs and the corresponding cut surfaces of the elongate main body 130. When the surgical thread 100 is cut in the undrawn state, the barbs 140 can have different geometries depending on the chosen cutting angle θ. Barbs 140 produced by cutting angles θ≧30 degrees, for example, (cf. FIG. 1d) generally have a more developed back side than barbs 140 produced by smaller cutting angles θ. The built-up configuration of the backs of the barbs is preferably due to a build-up 150 of material in the form of a material accumulation.

FIG. 1e is a schematic plan view of another example of the thread 100. The barbs 140 are formed on the surface of the thread 100 with a small angular offset as well as with small intervals between them. In each case two adjacent barbs 140 together form one barb having a twin-tip configuration (“double-acting” barb). As indicated in FIG. 1e (dashed barb 140), a spiral-shaped or helical type arrangement of the barbs 140 on the surface of the thread 100 may be produced in this manner.

FIG. 2a is a schematic view of thread 200. The thread 200 has a polymeric core 210 and a polymeric sheath 220 surrounding the core 210 (sheath-core construction). The thread has an elongate main body 230 from which individual barbs 240-243 project. The barbs 240-243 have an offset or staggered bidirectional arrangement on the main body 230. For one half of the thread 200, axially spaced barbs 240 are arranged about 180 degrees, in the circumferential direction, from and offset with respect to the barbs 242. Similarly, for the other half of the thread 200, the barbs 241 are likewise arranged about 180 degrees, in the circumferential direction, from and offset with respect to the barbs 243. The barbs 240 and 241 and the barbs 242 and 243 are each arranged bidirectionally relative to each other. The thread 200 can also be characterized by a cut surface angle γ. The cut surface angle γ is to be understood as the angle measured from the cut surfaces of the elongate main body 230 to the uncut outer surface of the elongate main body 230.

FIG. 2b is a schematic view of a cross-sectional surface along an imaginary line IIb-IIb of the example described in FIG. 2a of thread 200.

FIG. 3a is a schematic view of thread 300. The thread 300 has a polymeric core 310 and a polymeric sheath 320 surrounding the core 310 (sheath-core construction). The thread 300 further has an elongate main body 330 from which individual barbs 340-345 protrude. The barbs 340-345 have an offset or staggered bidirectional arrangement on the main body 330. For one half of the suture, axially spaced barbs 340 are arranged about 120 degrees, in the circumferential direction, from and offset/stacked with respect to the barbs 342, which in turn are arranged about 120 degrees, in the circumferential direction, from and offset with respect to the axially spaced barbs 344. Consequently, the barbs 344 are similarly arranged about 120 degrees, in the circumferential direction, from and offset with respect to the barbs 340. The same applies mutatis mutandis to the other half of the thread 300 with regard to the barbs 341; 343 and 345. The barbs 340 and 341, the barbs 342 and 343 and the barbs 344 and 345 are each arranged bidirectionally relative to each other.

FIG. 3b shows a schematic view of a cross-sectional surface along an imaginary line IIIb-IIIb of the example described in FIG. 3a of thread 300.

FIG. 4a is a schematic view of thread 400, the ends of which lie side by side forming a loop 460 and are attached to a surgical needle 470. There are no barbs in the region of the loop 460, whereas the other regions of the thread 400 have barbs 440 which protrude from a main body 430. The barbs 440 are arranged, in the straightened state of the thread 400 180 degrees, in the circumferential direction of the thread 400, from and offset with respect to each other. After formation of the loop, the barbs 440 face unidirectionally in the direction of the loop 460. The illustrated combination of surgical thread 400 and surgical needle 470 is particularly useful for knotless wound closure. The formation of a loop provides an advantageous way of producing a first secure retention point for closing a wound by threading the thread 400 through the loop 460 (FIG. 4b). Starting from this first retention point, a wound is stitched closed with the thread 400, with the barbs 440 anchoring themselves in the tissue region to be closed and thereby constituting additional points of retention (FIG. 4c). For clarity, the barbs are not shown in FIGS. 4b and 4c.

### EXAMPLES

### Example 1

### Producing a Surgical Thread Having a Sheath-Core Construction

A surgical thread having a sheath-core construction was produced using Monosyn® (triblock terpolymer of glycolide, trimethylene carbonate and s-caprolactone) as thread core polymer and poly(para-dioxanone) (PDO) as sheathing polymer in a volume ratio of 36:64. Production took place with the aid of a bicomponent monofilament range consisting of a twin-screw extruder, a single-screw extruder and also a bicomponent spinning head. The processing conditions employed were as follows:

| | Extruder 1 | Extruder 2 |
|---|---|---|
| | (Monosyn) | (PDO) |
| Zone 1-3 temperature [° C.] | 190/205/215 | 140/160/160 |
| Melt line temperature [° C.] | 215 | 180 |
| Spinning pump [ccm/rev.] | 0.25 | 0.25 |
| Spinning pump speed [rpm] | 12.2 | 21.8 |

| | |
|---|---|
| Spinning head temperature [° C.] | 210 |
| Die diameter [mm] | 2.0 |
| Quench bath temperature [° C.] | 20 |
| Air gap [cm] | 3 |
| Withdrawal speed [m/min] | 5 |
| Monofilament diameter [mm] | 1.32 |
| Sheath thickness [mm] | 0.24 |

The monofilament was subsequently drawn in two stages using the following process parameters:
Septett 1 delivery speed [m/min]: 5.0
Through oven 1 temperature [° C.]: 30
Septett 2 [m/min]: 22.5
Through oven 2 temperature [° C.]: 130
Septett 3 [m/min]: 24.3
Overall draw ratio: 4.86

After drawing, the monofilament had an overall diameter of about 0.60 mm and a sheath layer thickness of about 0.11 mm. Linear tensile strength was about 123 N with elongation at break being 38.7%.

### Example 2

### Producing a Sheath-Core Bicomponent Monofilament with Monosyn® as Core Polymer and Poly(Para-Dioxanone) (PDO) as Sheath Polymer in a Volume Ratio of 16:84

Example 1 was repeated except that the spinning pump speed was 5.4 rpm for extruder 1 (Monosyn®) and 28.6 rpm for extruder 2 (PDO). Monofilament diameter in the undrawn state was again about 1.32 mm, but sheath layer thickness was 0.40 mm. After drawing of the monofilament (same conditions as in Example 1), the overall diameter decreased to 0.60 mm and sheath layer thickness was 0.18 mm. Linear tensile strength was 117 N combined with elongation at break being 42.0%.

### Example 3

### Mechanical Cutting of Barbs into Bicomponent Monofilaments

The bicomponent monofilaments produced and drawn as per Examples 1 and 2 were placed in grooves in a metal plate which were 0.5 mm (monofilament produced as per Example 1) and 0.43 mm (monofilament produced as per Example 2) deep and 0.65 mm wide, and clamped tight. A microtome was used to cut into the monofilament along an inclined guide plane at an angle of 25 degrees between the blade and the monofilament surface, until the microtome came to rest on the metal plate. This produced cut depths (measured perpendicularly to the monofilament surface) of 0.10 mm (in the case of the monofilament produced as per Example 1) and 0.17 mm (in the case of the monofilament produced as per Example 2). Cut lengths were 0.24 mm (in the case of the monofilament produced as per Example 1) and 0.40 mm (in the case of the monofilament produced as per Example 2). With both monofilaments, the cuts were each limited to the sheath material. The cuts were spaced 0.8 mm apart.

### Example 4

### Thermal Cutting of Barbs into a Bicomponent Monofilament

The monofilament produced and drawn as per Example 2 was placed in a 0.35 mm deep and 0.65 mm wide groove in a Teflon plate and clamped tight. A fine wire about 35 μm in diameter was clamped into the insulated terminals of a fork-shaped device equipped with a handle. The fine wire was electrically connected to a controllable transformer, such that it could heat up as a function of the applied voltage. Preliminary tests showed that a voltage of about 5 volts was sufficient to heat the fine wire to a temperature sufficient to melt PDO but not Monosyn®.

The heated fine wire was then guided via a device to cut into the monofilament, and after the maximum depth of cut was reached back out again, at an angle of about 25 degrees. Although the monofilament protruded out of the groove by more than the thickness of the sheath layer, the core remained uncut. Further cuts were applied at a spacing of about 1 mm.

### Example 5

### Comparative Example for Mechanical Cutting of Barbs into a Drawn One-Component PDO Monofilament

A drawn PDO monofilament having a diameter of 0.60 mm was cut as described under Example 3 in grooves 0.50 mm and 0.43 mm deep to form barbs. The barbs had comparable geometries to the barbs produced under Example 3. However, the monofilament proved to have a distinctly higher flexural stiffness than the monofilaments described in Example 3.

### Example 6

### Comparative Example for Mechanical Cutting of Barbs into an Undrawn One-Component PDO Monofilament with Subsequent Drawing

An undrawn PDO monofilament having a diameter of 1.30 mm was placed, one hour after extrusion, into a groove 1.1 mm deep, clamped tight and cut as described under Example 7. Subsequently an attempt was made to draw the monofilament in a batch operation at room temperature. This was unsuccessful, however, since the monofilament would tear, the tears starting from the cuts, before any significant drawing had taken place. Regular drawing was only possible at a drawing temperature of about 48° C. The barbed monofilament obtained in this way proved to have a distinctly higher flexural stiffness than the bicomponent types of Example 7.

### Example 7

### Mechanical Cutting of Barbs into Undrawn Bicomponent Monofilaments with Subsequent Drawing

Undrawn monofilaments produced as per Examples 1 and 2 were each aged for 30 minutes. This reduced the tackiness of the previously amorphous PDO sheath and its crystallinity increased.

Monofilament pieces 30 cm in length were then placed in a groove having a depth of 1.1 mm (Example 1) or 0.95 mm (Example 2) and a width of 1.35 mm, clamped tight and cut as described under Example 3. The cut angle was about 30 degrees. Further cuts into the monofilament pieces were made at a spacing of 0.5 mm.

This was followed by batchwise drawing in a first step at room temperature. Unlike Example 6, thread rupture did not occur through tearing starting from the respective cut ends. Drawing at 48° C. was also possible. In a second step, setting was carried out at 90° C. in a circulating air oven, in the clamped state. In contrast to cutting in the drawn state, the barbs deployed to higher elevation as a result of this process.

### Example 8

### Producing a Bicomponent Monofilament have a Sheath-Core Construction Using Polypropylene (PP) as Core Polymer and Polyethylene Terephthalate (PET) as Sheathing Polymer in a Volume Ratio of 45:55

The materials used were polypropylene having an MFI of 2.8 and polylethylene terephthalate having an intrinsic viscosity of 0.9 dl/g.

| | Extruder 1 (PP) | Extruder 2 (PET) |
|---|---|---|
| Zone 1-3 temperature [° C.] | 230/230/230 | 260/280/280 |
| Melt line temperature [° C.] | 240 | 270 |
| Spinning pump [ccm/rev.] | 0.25 | 0.25 |
| Spinning pump speed [rpm] | 15.3 | 18.7 |

| | |
|---|---|
| Spinning head temperature [° C.] | 270 |
| Die diameter [mm] | 2.0 |
| Quench bath temperature [° C.] | 20 |
| Air gap [cm] | 3 |
| Withdrawal speed [m/min] | 5 |
| Monofilament diameter [mm] | 1.30 |
| Sheath thickness [mm] | 0.22 |

The monofilament was subsequently drawn in two stages using the following process parameters:
Septett 1 delivery speed [m/min]: 5.0
Through oven 1 temperature [° C.]: 80
Septett 2 [m/min]: 25.0
Through oven 2 temperature [° C.]: 140
Septett 3 [m/min]: 26.0
Overall draw ratio: 5.2

After drawing, the monofilament had an overall diameter of about 0.57 mm and a sheath layer thickness of about 0.1 mm. Linear tensile strength was about 121 N with elongation at break being 35.3%.

### Example 9

### Producing a Bicomponent Monofilament Having a Sheath-Core Construction Using Polyethylene Terephthalate (PET) as Core Polymer and Polypropylene (PP) as Sheathing Polymer in a Volume Ratio of 16:84

The bicomponent monofilament was produced in essentially the same way as in the process described in Example 8, except that the spinning pump speed was 5.4 rpm for extruder 1 (PP) and 28.6 rpm for extruder 2 (PET). Monofilament diameter in the undrawn state was 1.30 mm, the layer thickness of the polypropylene sheath was 0.39 mm. After drawing, carried out under the conditions recited in Example 8, the overall diameter decreased to 0.57 mm. Sheath layer thickness after drawing was 0.17 mm. Linear tensile strength was found to be 128 N with elongation at break equal to 29.7%.

### Example 10

### Mechanical Cutting of Barbs into an Undrawn Bicomponent Monofilament

An undrawn bicomponent monofilament produced as per Example 8 was placed into a 1.1 mm deep and 1.35 mm wide groove in a metal plate and clamped tight. Thereafter, a microtome was used along an inclined guide plane to cut into the monofilament at an angle of 30 degrees between the blade and the monofilament surface until the microtome came to rest on the metal plate. This resulted in a cut depth, measured perpendicularly to the monofilament surface, of 0.20 mm, which was restricted to the sheath material.

The monofilament was then batch drawn on a hot metal rail at 80° C., causing the barbs to deploy to a distinctly upright position. The barbs proved to be very stiff and pierced effectively into biological tissue without buckling. The monofilament itself turned out to be distinctly slacker in flexural stiffness than a PET one-component monofilament of the same gauge.

### Example 11

### Thermal Cutting of Barbs into a Monofilament Produced as Per Example 9

A monofilament produced as per Example 9 was placed into a 0.35 mm deep and 0.65 mm wide groove in a Teflon plate and subsequently clamped tight. To produce the barbs, a fine wire 35 μm in diameter was clamped into the insulated terminals of a fork-shaped device equipped with a handle. The fine wire was electrically connected to a controllable transformer, such that it could be heated up as a function of the applied voltage. Preliminary tests had found that a voltage of 6.5 volts was sufficient to heat the fine wire to a temperature sufficient to melt polypropylene, but not polyethylene terephthalate.

The monofilament was then cut with the electrically heated fine wire at an angle of 25 degrees via a guiding device. After the maximum depth of cut was reached, the fine wire was guided back out again. The monofilament protruded from the groove to an extent greater than equal to the thickness of the sheath layer. Yet the core of the monofilament remained uncut. Further cuts were applied at a spacing of 1 mm.

1. A surgical thread comprising a polymeric core and a polymeric sheath surrounding the core, wherein the sheath includes barbs for anchoring in human and/or animal tissues.

2. The surgical thread according to claim 1, wherein the barbs have a barb cut depth equal to not more than the thickness, of the polymeric sheath.

3. The surgical thread according to claim 1, wherein the barbs have a barb cut depth between 10 and 35% of the diameter of the surgical thread.

4. The surgical thread according to claim 1, wherein the polymeric core is more flexible than the polymeric sheath.

5. The surgical thread according to claim 1, wherein the polymeric core has a lower flexural modulus than the polymeric sheath.

6. The surgical thread according to claim 1, wherein the polymeric core has a flexural modulus between 200 and 2000 N/mm².

7. The surgical thread according to claim 1, wherein the polymeric sheath has a flexural modulus between 1000 and 10 000 N/mm².

8. The surgical thread according to claim 1, wherein the polymeric core has a higher melting point at least 20° C. higher than the melting point of the polymeric sheath.

9. The surgical thread according to claim 1, wherein the polymeric core is formed of an absorbable polymeric material having a glass transition point <30° C.

10. The surgical thread according to claim 1, wherein the polymeric core is formed of a block co- or terpolymer comprising at least one monomer selected from the group consisting of glycolide, lactide, trimethylene carbonate, ε-caprolactone and hydroxybutyric acid.

11. The surgical thread according to claim 1, wherein the polymeric core is formed of a nonabsorbable polymeric material selected from the group consisting of polyolefins, polyesters, polyamides, mixtures thereof, copolymers thereof and terpolymers thereof.

12. The surgical thread according to claim 1, wherein the polymeric sheath is formed of an absorbable polymeric material selected from the group consisting of poly(para-dioxanone), poly(ε-caprolactone), polylactide, polyglycolide, poly(trimethylene carbonate), poly(hydroxybutyric acid), mixtures thereof, copolymers thereof and terpolymers thereof.

13. The surgical thread according to claim 1, wherein the polymeric sheath is formed of a nonabsorbable polymeric material selected from the group consisting of polypropylene, polyethylene terephthalate, polyamide, mixtures thereof and copolymers thereof.

14. The surgical thread according to claim 1, wherein the thread has a linear tensile strength between 100 and 700 N/mm² based on a thread diameter without barbs.

15. The surgical thread according to claim 1, wherein the thread has an elongation at break between 15 and 100%.

16. The surgical thread according to claim 1, wherein a proportion of overall volume of the thread which is accounted for by the polymeric sheath is between 34% and 90% by volume.

17. The surgical thread according to claim 1, wherein the thread has a thickness between 0.2 and 1.2 mm.

18. The surgical thread according to claim 1, wherein the thread has a circular cross section and a proportion of a radius which is accounted for by the polymeric core is between 30 and 90%.

19. The surgical thread according to claim 1, wherein the thread comprises a surgical suture.

20. A surgical kit comprising at least one surgical needle and a surgical thread according to claim 1.

21. A method of forming a surgical thread according to claim 1, comprising forming a thread core polymer and a sheathing polymer into a thread having a polymeric sheath-core construction and barbs; and cutting the thread into the sheath.

22. The method according to claim 21, wherein the thread having the polymeric sheath-core construction is formed by coextruding the thread core polymer and the sheathing polymer.

23. The method according to claim 21, wherein the thread having the polymeric sheath-core construction is formed by sheathing extrusion, wherein the thread core polymer is sheathed by the sheathing polymer.

24. The method according to claim 21, wherein the barbs are cut into the sheath to a depth equal to not more than the thickness of the sheath.

25. The method according to claim 21, wherein the barbs are cut into the sheath to a depth between 10 and 35% of the diameter of the thread.

26. The method according to claim 21, wherein the barbs are cut into the thread in a drawn state of the thread.

27. The method according to claim 21, wherein the barbs are cut into the thread in an undrawn state of the thread and the thread is drawn thereafter.

28. The method according to claim 21, wherein the thread core polymer has a melting point at least 20° C. higher than the melting point of the sheathing polymer.

29. The method according to claim 21, wherein the barbs are cut into the sheath thermally in a temperature range below the melting point of the thread core polymer.

30. The method according claim 21, wherein the barbs are mechanically cut into the sheath by at least one cutting blade.

31. The method according to claim 21, wherein the barbs are cut into the sheath by a laser.

32. (canceled)
